(19) United States
(12) Patent Application Publication
(10) Pub. No.: US 2024/0336670 A1
(43) Pub. Date: Oct. 10, 2024

(54) PURIFICATION OF LIRAGLUTIDE

(71) Applicant: BIOCON LIMITED, Bangalore (IN)

(72) Inventors: Ganesh RAMACHANDRAN, Bangalore (IN); Nitin Sopanrao PATIL, Bangalore (IN); Onkar SANTAN, Bangalore (IN); Baraneedharan Gnana SAMBANDAM, Tamil Nadu (IN); Kruthi Sathish BASTIKOPPA, Karnataka (IN)

(21) Appl. No.: 18/682,254

(22) PCT Filed: Aug. 9, 2022
(86) PCT No.: PCT/IB2022/057409

§ 371 Date: Feb. 8, 2024

(30) Foreign Application Priority Data
Aug. 10, 2021 (IN) 202141036153

(51) Int. Cl. C07K 14/605; C07K 1/36

(52) U.S. Cl. CPC C07K 14/605; C07K 1/36

(57) ABSTRACT

The present invention provides for novel and improved liraglutide precursor purification processes of liraglutide precursor using selective unit operation steps and selective pH gradients in the reversed phase-high performance liquid Chromatography. for purifying crude liraglutide precursor from closely related impurities.

### RELATED APPLICATION

This application claims the benefit of priority of our Indian patent applications IN 202141036153 filed on Aug. 10, 2021, which is incorporated herein by reference.

### TECHNICAL FIELD

The present invention relates to a method for purifying crude GLP-1 analogue, precursor of Liraglutide in particular which is represented by the Formula-I.

### BACKGROUND AND PRIOR ART OF THE DISCLOSURE

Liraglutide (VICTOZA®) is a glucagon-like peptide-1 (GLP-1) receptor agonist indicated as an adjunct to diet and exercise to improve glycemic control in adults with type 2 diabetes mellitus.

Liraglutide, is a long acting analogue of the naturally occurring human glucagon like peptide-1 (GLP-1 (7-37)) in which lysine at position 34 has been replaced with arginine and palmitoyl group has been attached via glutamoyl spacer to lysine at position 26.

Liraglutide (VICTOZA®), developed by Novo Nordisk got initial approval in United

States in 2010 as subcutaneous injection. Liraglutide due to its long peptide chain and high hydrophobicity due to palmitoyl group is highly difficult to purify.

Several attempts for purification of GLP-1 analogues including Liraglutide have been reported in the past.

Journal of Medicinal Chemistry 43, 1664-1669, 2000 discloses a purification process of Liraglutide by reversed phase-high performance liquid Chromatography (RP HPLC) using a cyanopropyl column (Zorbax 300SB-CN) and a standard acetonitrile/TFA system.

The method as disclosed above results in a reduced purification yield of 35%.

WO2013117135 discloses a purification process of Liraglutide by RP HPLC using Isopropyl alcohol/TFA system.

The method as disclosed involves multiple purification steps involving 3 RP HPLC operations, which is a laborious process.

GLP-1 peptides are produced either by synthetic or by recombinant approach often have closely related impurities that are difficult to separate on RP-HPLC. These impurities are either isomeric or deletion/addition based impurities that have similar characteristics like the parent molecule. These closely related impurities pose a challenge in purification.

It is well-known that the use of RP-HPLC is limited for the separation and identification of complex mixtures having components with large variation in pKa values. Thus, resolution of closely eluting impurities has always been challenging in chromatographic purification.

Also, when the Liraglutide precursor produced by recombinant approach, challenges of purification involves the separation of the cells and fermentation media components from the precursor, removal of the host cell proteins and host cell DNA and related impurities, impurities with additional hexose units and deletion impurities.

It was observed in the present invention that the resolution the above said impurities needed a purification process comprising various steps to achieve the desired purity.

### SUMMARY OF THE INVENTION

Aspects of the present application provides processes for purification of liraglutide precursor.

One aspect of the present invention discloses a method for purifying crude recombinant liraglutide precursor, the method comprising:
a. subjecting the fermentation broth to microfiltration;
b. subjecting the product of step a) to diafiltration;
c. solubilizing the product of step b), followed by centrifugation;
d. subjecting the product of step c) to depth filtration step;
e. the filtered supernatant from step d) is subjected to cation exchange chromatography purification step
f. subjecting the product of step e) to a reversed phase high pressure liquid chromatography (RP-HPLC); and
g. Isolating the purified liraglutide precursor.

Another aspect of the present invention discloses a method for purifying liraglutide precursor, wherein microfiltration is performed at pH 3.0 to 6.0.

Another aspect of the present invention discloses a method for purifying liraglutide precursor, wherein diafiltration is performed at pH 3.0 to 6.0.

Another aspect of the present invention discloses a method for purifying liraglutide precursor, wherein solubilizing is performed by addition of Urea.

Another aspect of the present invention discloses a method for purifying liraglutide precursor, wherein mobile phase gradient is a buffer with a pH range of 3.0 to 5.0.

Another aspect of the present invention discloses a method for purifying liraglutide precursor, wherein the buffer is selected from Glycine-HCL buffer, citrate buffer, acetate buffer, citrate-phosphate buffer, succinate buffer, maleate buffer.

Another aspect of the present invention discloses a method for purifying crude recombinant liraglutide precursor, the method comprising:
a. subjecting the fermentation broth to microfiltration at pH 3.0 to 6.0;
b. subjecting the product of step a) to diafiltration at pH 3.0 to 6.0;
c. solubilizing the product of step b) using Urea, followed by centrifugation;
d. subjecting the product of step c) to Depth filtration step;
e. the filtered supernatant from step d) is subjected to cation exchange chromatography purification step
f. subjecting the product of step e) to a reversed phase high pressure liquid chromatography (RP-HPLC); and
g. Isolating the purified liraglutide precursor.

Wherein mobile phase gradient is a buffer with a pH range of 3.0 to 5.0.

Advantages of present invention:
1. The Liraglutide precursor is expressed extracellularly, there is no lysis involved.
2. Initial volume of broth is reduced considerably (3.5-4 times) credited to the usage of the microfiltration/diafiltration operation.
3. No solvent is used in the capture chromatography, viz. cation exchange chromatography step, thus process is economical.
4. Purified precursor has a purity of >98% that is taken up for acylation.
5. Overall yield of the process till precursor purification is significantly high and economically viable, viz. 45%. The yield of the acylated liraglutide purification is also high, >60% with purity >99.5%.

Each step of the process disclosed herein are contemplated both in the context of the multistep sequences described and individually.

### BRIEF DESCRIPTION OF THE FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1: Illustrates the preparative profile of Cat-ion exchange chromatographic step prepared according to Example 1.

FIG. 2: Illustrates the preparative profile of RP-HPLC I step, prepared according to Example 2.

FIG. 3: Illustrates the SDS-PAGE image which gives the comparative purity profile across different unit operations till the purified precursor according to Example 2.

FIG. 4: Illustrates the preparative profile of RP-HPLC II step, prepared according to Example 4.

FIG. 5: Illustrates the preparative profile of RP-HPLC III step, prepared according to Example 5.

FIG. 6: Illustrates the SDS-PAGE image which of final purified Drug Substance (Silver staining) according to Example 6.

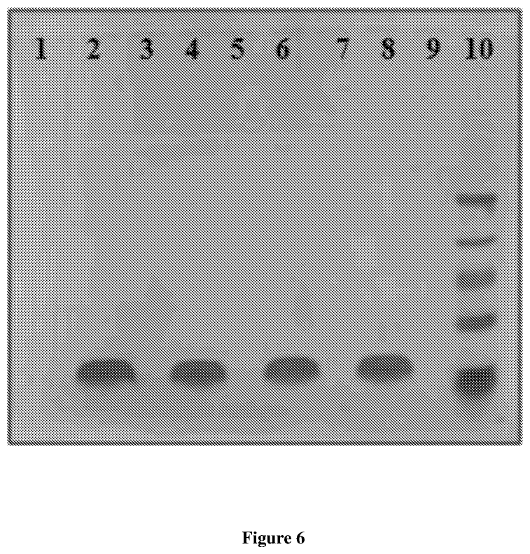

FIG. 7: Illustrates the total ion chromatogram (TIC) overlay profile of CEX pellet vs RP pellet according to Example 6.

### DETAILED DESCRIPTION OF THE INVENTION

The embodiments of the present invention are further described using specific examples herein after. The examples are provided for better understanding of certain embodiments of the invention and not, in any manner, to limit the scope thereof. Possible modifications and equivalents apparent to those skilled in the art using the teachings of the present description and the general art in the field of the invention shall also form the part of this specification and are intended to be included within the scope of it.

### EXAMPLES

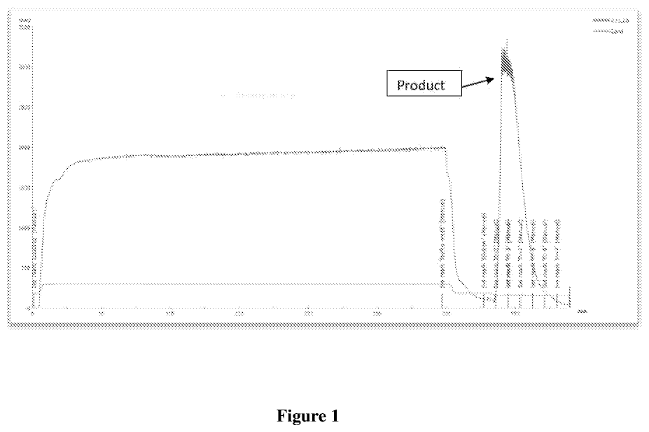

Example 1:530 kg of fermentation broth having a titer of 0.4 g/L was subjected to MF and DF followed by urea solubilization and centrifugation. The HPLC purity of the precursor determined at the end of centrifugation was 8%. This was then loaded on a pre-equilibrated cation exchange (CEX) column, followed by washing and a pH-based elution. The pool purity of the CEX fractions was found to be 70-75%. The pH of the CEX fractions was adjusted to 3.5-5.5 to yield a CEX pellet. Detection wavelength was kept at 280 nm. The chromatographic temperature was kept at 25° C. The preparative chromatogram is as shown in FIG. 1.

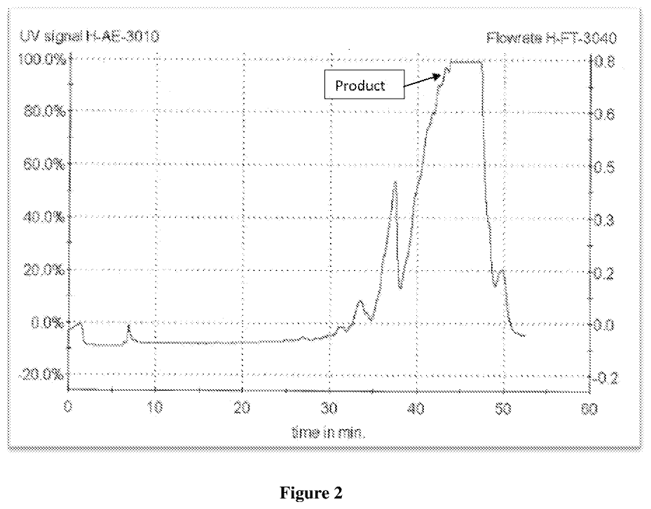

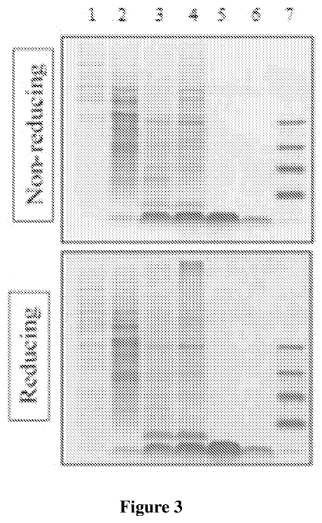

Example 2: The CEX pellet obtained from Example 1 was then purified on RP-HPLC-I using a 2.4L C8 column. The bound precursor was eluted using a step gradient of the mobile phase (A: Acetate buffer; B: ACN). Detection wavelength was kept at 280 nm. The chromatographic temperature was kept at 25° C. The preparative chromatogram is as shown in FIG. 2. Fractions having purity >97% was concentrated under vacuum followed by iso-electric point precipitation. The suspension was centrifuged to yield the precipitate of purified precursor. The precipitate was washed with water, centrifuged, and stored at −20° C. had a HPLC purity of >98%. The SDS-PAGE image shown in FIG. 3 gives the comparative purity profile across different unit operations till the purified precursor.

| Lanes | Samples details |
|---|---|
| 1 | Broth |
| 2 | Centrifuged supernatant |
| 3 | CEX Pool |
| 4 | CEX Pellet |
| 5 | RP Pool |
| 6 | Standard precursor |
| 7 | Protein Marker |

Example 3: 59 g of the purified precursor having a purity of 98.3% obtained from Example 2 was subjected to the acylation step. The acylation yield was >70% and the crude liraglutide obtained had an assay of >65% with a HPLC purity of 77%.

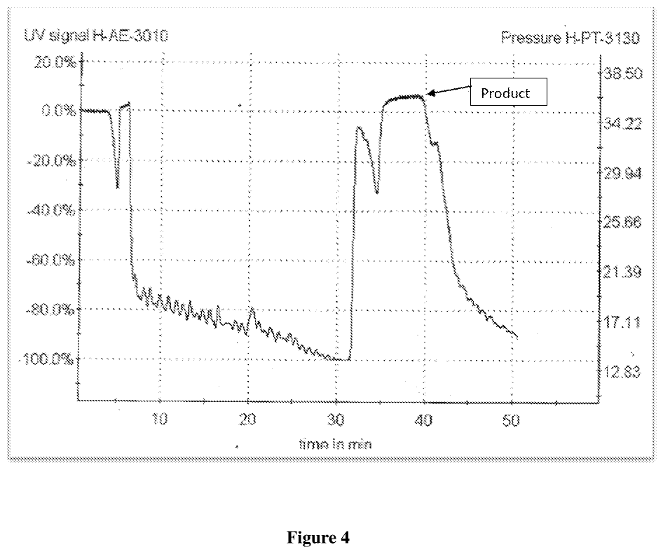

Example 4: The acylated crude was dissolved in equilibration buffer having pH of 2.0-4.0 and loaded on a 2.4L pre-equilibrated C8 column. The bound product was eluted using a gradient (A: Equilibration buffer; B: ACN: IPA) and analysed for HPLC purity and product content. The pH of the fractions was diluted using phosphate buffer and stored at 2-8° C. Finally, fractions are pooled to achieve pool purity >99% with step yield of 75-80%. Detection wavelength was kept at 215 nm. The chromatographic temperature was kept at 25°° C. The preparative chromatogram is as shown in FIG. 4.

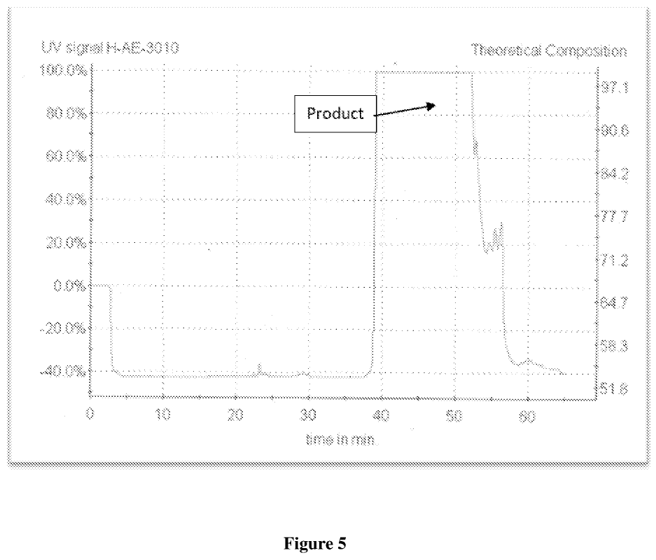

Example 5: RP-HPLC-II elution pool was further purified by reversed phase high pressure chromatography (RP-HPLC-III). The pH of RP-2 pool was adjusted to 6.5-8.0, diluted, and loaded onto pre-equilibrated 2.4 L, C8 column. The bound product was eluted using a gradient (A: Equilibration buffer; B: ACN) and analysed for HPLC purity and product content. Detection wavelength was kept at 215 nm. The chromatographic temperature was kept at 25° C. The preparative chromatogram is as shown in FIG. 5.

The pH of the fractions was diluted using citrate buffer and stored at 2-8° C. Finally, fractions were pooled to achieve pool purity ≥99.5% with step yield of >90%. The pool was centrifuged to separate the pellet followed by water washing and the purified pellet is isolated. The isolated pellet is lyophilized to yield the purified Liraglutide.

| Lanes | Samples details |
|---|---|
| 2 | Batch 1 |
| 4 | Batch 2 |
| 6 | Batch 3 |
| 8 | RLD-Victoza |
| 10 | Molecular weight marker |
| | (1.4 to 26.6 kDa) |

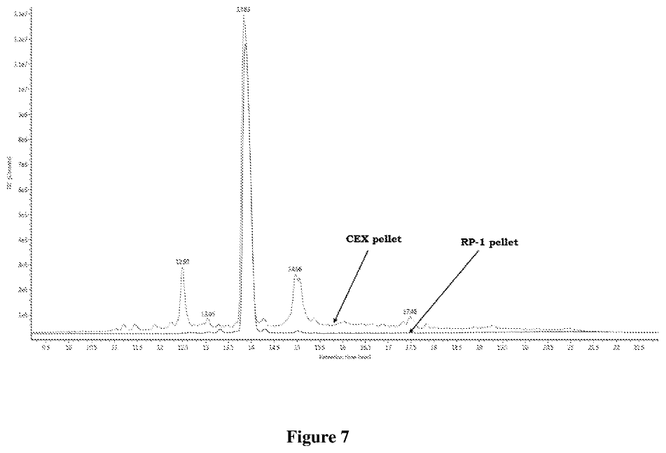

Example 6:12170 kg of fermentation broth having a titer of 0.4 g/L was subjected to Micro Filtration (MF) and Diafiltration (DF) followed by urea solubilization and centrifugation. The HPLC purity of the precursor determined at the end of centrifugation was 8%. This was then loaded on a pre-equilibrated cation exchange column, followed by washing and a pH-based elution. The pool purity of the CEX fractions was found to be 68-73%. The pH of the CEX fractions was adjusted to 3.5-5.5 to yield a CEX pellet, which had a purity of 78-80%. Detection wavelength was kept at 280 nm. The chromatographic temperature was kept at 25°° C. The preparative chromatogram was similar to shown in FIG. 1. The CEX pellet was then purified on RP-HPLC-I using a 18 L C8 column. The bound precursor was eluted using a step gradient of the mobile phase (A: Acetate buffer; B: ACN). Detection wavelength was kept at 280 nm. The chromatographic temperature was kept at 25° C. The preparative chromatogram was similar to as shown in FIG. 2. Fractions having purity >93% was concentrated under vacuum followed by iso-electric point precipitation. The suspension was centrifuged to yield the precipitate of purified precursor. The precipitate was washed with water, centrifuged, and stored at-20° C. had a HPLC purity of >98%. The CEX pellet and RP pellet were analyzed by High resolution Mass spectrometry (HR-MS) to understand the identity of the impurities. The total ion chromatogram (TIC) overlay profile of CEX pellet vs RP pellet is as shown in FIG. 7. Based on the HR-MS profile of CEX pellet, there was addition of hexose units (mono-hexose, dihexose, trihexose, tetrahexose) at 0.90 RRT (12.50 mins in TIC), some deletion impurities at 1.03-1.16RRT (14.2 mins to 16 mins in TIC) and High molecular weight protein (HMWP) impurities at 1.24-1.39RRT (17-19.2 mins in TIC). Most of these impurities are resolved in RP-HPLC I step and only minor levels of deletion impurities are present in the RP-HPLC pellet which was >98% pure.

Example 7: The RP-1 pellet obtained from Example 6 was subjected to the acylation step. The acylation yield was 60% and the crude liraglutide obtained had an assay of 70% with a HPLC purity of >80%. This acylated crude was then purified on a 18L C8 column. The acylated crude was dissolved in equilibration buffer having pH of 2.0-4.0 and loaded on the pre-equilibrated C8 column. The bound product was eluted using a gradient (A: Equilibration buffer; B: ACN: IPA) and analysed for HPLC purity and product content. The pH of the fractions was diluted using phosphate buffer and stored at 2-8° C. Finally, fractions were pooled to achieve pool purity >99% with step yield of 80-85%. Detection wavelength was kept at 215 nm. The chromatographic temperature was kept at 25° C. The preparative chromatogram is similar to as shown in FIG. 4.

Example 8: The pH of elution pool from example 7 was adjusted to 6.5-8.0, diluted, and loaded onto pre-equilibrated 18 L, C8 column. The bound product was eluted using a gradient (A: Equilibration buffer; B: ACN) and analysed for HPLC purity and product content. Detection wavelength was kept at 215 nm. The chromatographic temperature was kept at 25°° C. The preparative chromatogram is similar to as shown in FIG. 5. The pH of the fractions was diluted using citrate buffer and stored at 2-8° C. Finally, fractions were pooled to achieve pool purity >99.5% with step yield of >93%. The pool was centrifuged to separate the pellet followed by water washing and the purified pellet was isolated. The isolated pellet was lyophilized to yield the purified Liraglutide.

1. A method for purifying crude recombinant liraglutide precursor, the method comprising:
a. subjecting the fermentation broth to microfiltration;
b. subjecting the product of step a) to diafiltration;
c. solubilizing the product of step b), followed by centrifugation;
d. subjecting the product of step c) to depth filtration step;
e. the filtered supernatant from step d) is subjected to cation exchange chromatography purification step;
f. subjecting the product of step e) to a reversed phase high pressure liquid chromatography (RP-HPLC); and
g. Isolating the purified liraglutide precursor.

2. The method of claim 1, wherein microfiltration is performed at pH 3.0 to 6.0.

3. The method of claim 1, wherein diafiltration is performed at pH 3.0 to 6.0.

4. The method of claim 1, wherein solubilizing is performed by addition of Urea.

5. The method of claim 1, wherein mobile phase gradient is a buffer with a pH range of 3.0 to 5.0.

6. The method of claim 5, wherein the buffer is selected from Glycine-HCL buffer, citrate buffer, acetate buffer, citrate-phosphate buffer, succinate buffer, maleate buffer.

7. A method for purifying crude recombinant liraglutide precursor, the method comprising:
a. subjecting the fermentation broth to microfiltration at pH 3.0 to 6.0;
b. subjecting the product of step a) to diafiltration at pH 3.0 to 6.0;
c. solubilizing the product of step b) using Urea, followed by centrifugation;
d. subjecting the product of step c) to Depth filtration step;
e. the filtered supernatant from step d) is subjected to cation exchange chromatography purification step;
f. subjecting the product of step e) to a reversed phase high pressure liquid chromatography (RP-HPLC); and
g. Isolating the purified liraglutide precursor. Wherein mobile phase gradient is a buffer with a pH range of 3.0 to 5.0.
